(19) United States
(12) Patent Application Publication
(10) Pub. No.: US 2002/0107388 A1
(43) Pub. Date: Aug. 8, 2002

(54) Methods of identifying and monitoring disease-associated T cells

(76) Inventor: Arthur A. Vandenbark, Portland, OR (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/853,830

(22) Filed: May 10, 2001

Related U.S. Application Data
(60) Provisional application No. 60/203,984, filed on May 12, 2000.

(51) Int. Cl. C12Q 1/68; C07H 21/04; C12P 19/34

(52) U.S. Cl. 536/24.3; 435/6; 435/91.2

(57) ABSTRACT

The invention provides a method of identifying a T cell receptor (TCR) variable (V) gene expressed by target T cells in an individual. The method is practiced by determining expression of one or more TCR V genes by activated T cells from the individual, and determining regulatory activity elicited in response to one or more TCR V peptides by T cells from the individual. A TCR V gene that is preferentially expressed, whose corresponding TCR V peptide elicits low T cell regulatory activity, is identified as a V gene expressed by target T cells.

This application claims priority to provisional application Ser. No. 60/203,984, filed May 12, 2000, which is incorporated herein by reference in its entirety.

This invention was made with United States Government support under grant numbers NS23221 and NS23444, awarded by the National Institutes of Health. The U.S. Government has certain rights in this invention.

### BACKGROUND OF THE INVENTION

1. Field of the Invention

This invention relates to the fields of medicine and immunology and, more specifically, to methods of identifying and monitoring disease-associated T cells.

2. Background Information

Autoimmune disorders affect about 5% of the human population, often causing chronic, debilitating illnesses. Although all individuals have immune cells that potentially react with antigens present on their own tissues, these autoreactive cells are normally held in check by complex and currently poorly understood regulatory mechanisms. In individuals who develop autoimmune disease, these regulatory mechanisms are proposed to be somehow defective, which allows autoreactive cells to mount an immunological attack against host tissues.

Animal models have aided in understanding the mechanisms underlying autoimmune diseases. For example, experimental allergic encephalomyelitis (EAE) is an autoimmune disease of the central nervous system that can be induced in mice and rats by immunization with myelin basic protein (MBP). Histologically and clinically, EAE resembles multiple sclerosis (MS) in humans. EAE is mediated by T cells having specificity for MBP, as evidenced by the ability of MBP-reactive T cells to induce EAE when adoptively transferred to healthy hosts. Analysis of the antigen-binding receptor, or T cell receptor (TCR), expressed by MBP-reactive T cells has generally revealed that these T cells express a limited number of TCR V alpha (VA) and TCR V beta (VB) polypeptide chains.

The TCR is a heterodimeric cell surface glycoprotein present on the surface of T cells. The TCR exists in two forms, one consisting of an alpha chain and a beta chain, the second consisting of a gamma chain and a delta chain. Each TCR polypeptide chain is encoded by a genetic locus containing multiple discontinuous gene segments. These include variable (V) region gene segments, joining (J) region gene segments and constant (C) region gene segments. Beta and delta chains contain an additional element termed the diversity (D) gene segment. The TCR gene segments become rearranged during T cell maturation to form VJ or VDJ genes, which are then expressed as polypeptide chains. There are at least 50 different human Vα (or VA), 57-70 Vβ (or VB), 3 Vδ and 7Vγ gene segments, which are categorized into various families, with members of a family sharing substantial nucleotide and amino acid sequence identity.

EAE has successfully been prevented or treated by various methods that selectively target the TCR V gene present on encephalitogenic T cells. Such therapeutic methods include immunization with TCR V region peptides to induce an immune response against the autoreactive T cells, and administering anti-TCR V region antibodies to bind and either kill or inactivate the autoreactive T cells. Once the disease-associated TCR V genes are identified in humans, analogous immunotherapeutic methods that target T cells expressing these V genes are also expected to be effective.

However, human autoimmune diseases have proven to be more complex than experimental animal models, in part because there are numerous autoantigens implicated in human diseases, and human responses to different autoantigens depend on genetic factors. In certain studies, T cells from individuals with autoimmune disease that react to proposed autoantigens have been demonstrated to express a limited subset of V genes. However, the relevance of these T cells to the disease is as yet unclear, because the particular antigen used in assessing T cell reactivity is not necessarily involved in the etiology of the disease in that individual. Additionally, in certain studies, T cells obtained from the site of the pathology from individuals with autoimmune disease have been demonstrated to express a limited subset of V genes. Unfortunately, for most autoimmune diseases, and particularly for diseases that affect internal tissues, it is difficult or impossible to obtain samples of T cells from the relevant site. Additionally, the currently available methods of identifying TCR V gene usage do not take into account the regulatory mechanisms that may be acting in a particular individual to control the activity of the relevant T cells.

Therefore, there exists a need for an improved method of identifying disease-associated T cells in individuals, including both autoreactive T cells and regulatory T cells. Once the identity of these cells is known, appropriate, individualized therapies can be designed to prevent or treat the disease. The present invention satisfies this need and provides related advantages as well.

### SUMMARY OF THE INVENTION

The invention provides a method of identifying a T cell receptor (TCR) variable (V) gene expressed by target T cells in an individual. The method is practiced by determining expression of one or more TCR V genes by activated T cells from the individual, and determining regulatory activity elicited in response to one or more TCR V peptides by T cells from the individual. A TCR V gene that is preferentially expressed, whose corresponding TCR V peptide elicits low T cell regulatory activity, is identified as a V gene expressed by target T cells. Also provided are kits suitable for use in the method.

The invention also provides methods of monitoring the efficacy of a therapy for an autoimmune disease. The methods are practiced by identifying a TCR V gene expressed by target T cells in an individual with an autoimmune disease and either determining T cell regulatory activity elicited in response to the corresponding TCR V peptide after initiation of therapy, or determining expression of the V gene by activated T cells from the individual after initiation of therapy.

Also provided is a method of selecting a therapy for an autoimmune disease. The method is practiced by identifying a TCR V gene expressed by target T cells in an individual with an autoimmune disease and selecting a therapy that targets T cells expressing said TCR V gene.

### BRIEF DESCRIPTION OF THE DRAWINGS

FIG. 1 shows ELISPOT scattergram frequencies of IL-10 and IFNγ secreting peripheral blood mononuclear cells (PBMC) in multiple sclerosis patients (MS), MS patients who had previously been vaccinated with (Y49T) BV5S2-38-58 peptide (*), and healthy controls (HC) in response to A) BV5S2-38-58 peptide; B) (Y49T) BV5S2-38-58 peptide; C) BV6S1-38-58 peptide; and D) ConA.

FIG. 2 shows an analysis of frequencies of IL-10 secreting PBMC in a (Y49T)BV5S2-38-58 peptide-vaccinated MS patient in response to native or substituted BV5S2-38-58 peptide, as determined by ELISPOT. Time points shown are one week after booster injection, three weeks later just prior to a further booster injection, and one week after the further booster injection.

FIG. 3 shows BV peptide recognition and BV gene expression by activated memory T-cells from a healthy control (HC) (panels A-D) and an MS patient (panels E-H). Panels A and E show IL-10 responses of PBMC to BV CDR2 peptides and ConA as determined by ELISPOT. Panels B, C, F and G show FACS profiles of the indicated T cell populatons. Panels D and H show BV gene expression by the indicated presorted and sorted T cell populations as determined by RT-PCR.

FIG. 4 shows mean frequencies of IL-10 and IFNγ secreting PBMC in response to a panel of different Vα (VA) CDR2 region peptides (amino acids 38-58) in four unvaccinated, healthy individuals, as determined by ELI SPOT.

FIG. 5 shows mean frequencies of IL-10 and IFNγ secreting PBMC in response to a panel of different Vβ (VB) CDR2 region peptides (amino acids 38-58) in four unvaccinated, healthy individuals, as determined by ELISPOT.

FIG. 6 shows the frequency of IL-10 secreting PBMC in response to a panel of VA and VB peptides CDR2 region peptides (amino acids 38-58) in an individual with MS (left panel) and mean values from four normal individuals (right panel), as determined by ELISPOT.

### DETAILED DESCRIPTION OF THE INVENTION

The invention provides a method of identifying T cells that have escaped from normal regulatory control in an individual. These cells serve as appropriate targets for therapies aimed at preventing or treating autoimmune pathologies and are thus designated herein “target T cells.” Once the target T cells are identified, either these T cells, their specific regulators, or both, can be monitored over time to determine development or progression of an autoimmune disease. The target T cells and their specific regulators can also be monitored during the course of therapy to determine the efficacy of the therapy, and to determine whether or when it is appropriate to administer or re-administer a therapy. With this knowledge, therapies can be tailored for a particular individual so as to increase the likelihood of choosing an effective therapy, and to correspondingly decrease the likelihood of choosing an ineffective therapy that unnecessarily exposes the individual to harmful or unpleasant side effects.

The following model for autoimmune disease development arises from the disclosures herein. Unexpectedly, healthy individuals contain regulatory T cells specific for most expressed T cell receptor (TCR) variable (V) genes. These regulatory T cells are proposed to normally function to control the activity of T cells that express the corresponding V genes. In healthy individuals, potentially autoreactive T cells are held in check, in part, by these regulatory TCR V-specific T cells. However, in individuals that develop autoimmune disease, there is defective regulatory activity towards T cells that express certain V genes. In the presence of an autoantigen stimulus, this regulatory defect allows oligoclonal expansion of autoreactive T cells that express certain of these V genes, which leads to recruitment of other inflammatory T cells to the involved tissue, culminating in tissue damage.

The method of identifying the TCR V gene expressed by a target T cell population is practiced by a) determining expression of one or more T cell receptor (TCR) variable (V) genes by activated T cells from an individual, and b) determining regulatory activity elicited in response to one or more TCR V peptides by T cells from said individual. A TCR V gene that is preferentially expressed in step a), whose corresponding TCR V peptide elicits a low regulatory T cell response in step b), is identified as a V gene expressed by a target T cell population.

As shown in Example I, activated memory T cells from a patient with multiple sclerosis (MS) preferentially express the BV6S1 gene compared to unselected CD4+ T cells from the same patient, and regulatory activity (IL-10 secretion) in response to a corresponding BV6S1 peptide is low in comparison with a normal individual. Therefore, in this individual, T cells that express the BV6S1 gene are identified as “target T cells.” These target T cells, and their regulators, are relevant to the development, maintenance and/or progression of the disease, and thus a therapy should be chosen for this individual that causes a reduction in the number of activated BV6S1-expressing T cells, or an increase in the number of T cells that regulate BV6S1-expressing T cells, or both.

The method can be practiced to identify target T cells in an individual with an autoimmune disease, or in an individual predicted to be at risk of developing an autoimmune disease. Clinical and prognostic indicators of autoimmune diseases are well known in the art. Exemplary autoimmune diseases affecting mammals include rheumatoid arthritis (RA), juvenile oligoarthritis, collagen-induced arthritis, adjuvant-induced arthritis, Sjogren's syndrome, multiple sclerosis (MS), experimental autoimmune encephalomyelitis (EAE), inflammatory bowel disease (e.g. Crohn's disease, ulceritive colitis), autoimmune gastric atrophy, pemphigus vulgaris, psoriasis, vitiligo, type I diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia pernicious anemia, and the like.

The steps in the method, namely determination of V gene expression by activated T cells, and determination of regulatory activity in response to the corresponding V peptide, can be practiced either simultaneously, or sequentially in either order.

At least one of the TCR V genes and at least one of the TCR V peptides used in the method will “correspond” to each other. As used herein, the term “corresponding TCR V peptide” indicates that the peptide has the same, or substantially the same, amino acid sequence as a portion of the polypeptide encoded by the referenced V gene and elicits an immune response against the polypeptide encoded by the the referenced V gene.

It is not required that there be a one-to-one correspondence between the V genes and V peptides used in the method. For example, Example I exemplifies practice of the method by determining expression of a panel of more than 20 different VB genes (including BV6S1 and BV5S2), while determining regulatory activity in response to a smaller subset of corresponding TCR V peptides, namely BV6S1-38-58, BV5S2-38-58 and (Y49T) BV5S2-38-58.

As described above, one component of the mechanism underlying autoimmune disease is defective regulatory activity by T cells that control the proliferation and activity of autoreactive T cells that express particular TCR V genes. Defective regulation of T cells expressing particular TCR V genes can be evidenced by stimulating T cells with a particular TCR V peptide, and detecting abnormally low regulatory activity by T cells in response to that peptide.

As used herein, the term “regulatory activity” refers to a detectable property that correlates with Th2-type, anti-inflammatory T cell activity. The particular regulatory activity to detect in the method will depend on the type and sensitivity of the assay used, and can be chosen by the skilled person.

In one embodiment, the regulatory activity is expression of a regulatory cytokine. The term “regulatory cytokine” is intended to include Th2 cytokines such as interleukin-10 (IL-10), IL-4, IL-13, transforming growth factor beta (TGFβ), and other cytokines that are predominantly anti-inflammatory. Other cytokines that under appropriate conditions have anti-inflammatory effects include IL-S, TNF-α IL-9, IFNβ and IFNγ. An appropriate regulatory cytokine to assay in the methods of the invention can be determined by the skilled person.

A variety of methods can be used to detect and quantitate cytokine expression by T cells. For example, an immunospot assay, such as the enzyme-linked immunospot or “ELISPOT” assay, can be used. The immunospot assay is a highly sensitive and quantitative assay for detecting cytokine secretion at the single cell level. Immunospot methods and applications are well known in the art and are described, for example in Czerkinsky et al., J. Immunol. Methods 110:29-36 (1988); Olsson et al. J. Clin. Invest. 86:981-985 (1990); and EP 957359.

In general, the immunospot assay uses microtiter plates containing membranes that are precoated with a capture agent, such as an anti-cytokine antibody, specific for the cytokine to be detected. T cells of interest are plated together with a test immunogen, which in the invention method is a TCR V peptide. The T cells that respond to the immunogen secrete various cytokines. As the cytokine of interest is locally released by the T cells, it is captured by the membrane-bound antibody. After a suitable period of time the cell culture is terminated, the T cells are removed and the plate-bound cytokine is visualized by an appropriate detection system. Each cytokine-secreting T cell will ideally be represented as a detectable spot. The number of spots, and thus the number of T cells secreting the particular cytokine of interest, can be counted manually (e.g. by visualization by light microscopy) or by using an automated scanning system (e.g. an Immunospot Reader from Cellular Technology Ltd.). Examples I and II describe the use of an ELISPOT assay to quantitate and compare the number of regulatory T cells that secrete IL-10 (and/or IFNγ) in response to different TCR V peptides in different individuals.

Variations of the standard immunospot assay are well known in the art and can be used to detect cytokine secretion in the methods of the invention. For example, U.S. Pat. No. 6,218,132 describes a modified immunospot assay in which antigen-responsive T cells are allowed to proliferate in response to stimulation with the immunogen before detection of the cytokine of interest. This method, although more time-consuming, can be used to increase the sensitivity of the assay for detecting T cells present at a low frequency in the starting population.

Likewise, U.S. Pat. No. 5,939,281 describes an improved immunospot assay that uses a hydrophobic membrane instead of the conventional nitrocellulose membrane, to bind the cytokine capture reagent. This variation can be used to reduce the non-specific background and increase the sensitivity of the assay.

Other modifications to the standard immunospot assay that increase the speed of processing multiple samples, decrease the amount of reagents and T cells needed in the assay, or increase the sensitivity or reliability of the assay, are contemplated herein and can be determined by those skilled in the art.

Antibodies suitable for use in immunospot assays, which are specific for secreted cytokines, as well as detection reagents and automated detection systems, are well known in the art and generally are commercially available. Appropriate detection reagents are also well known in the art and commercially available, and include, for example, secondary antibodies conjugated to fluorochromes, colored beads, and enzymes whose substrates can be converted to colored products (e.g., horseradish peroxidase and alkaline phosphatase). Other suitable detection reagents include secondary agents conjugated to ligands (e.g. biotin) that can be detected with a tertiary reagent (e.g. streptaviden) that is detectably labeled as above.

Other methods for detecting and quantifying cytokine expression by T cells are well known in the art, and can be used as an alternative to immunospot assays in the methods of the invention. Such methods include the ELISA assay, which can be used to measure the amount of cytokine secreted by T cells into a supernatant (see, for example, Vandenbark et al., Nature Med. 2:1109-1115 (1996)). Alternatively, the expression of cytokine mRNA can be determined by standard immunological methods, which include RT-PCR and in-situ hybridization.

As an alternative to determining regulatory cytokine expression in response to TCR peptides, other regulatory activities that correlate with anti-inflammatory function of T cells can be assessed. These activities can include, for example, T cell proliferation, expression of proliferation markers (e.g. c-fos, c-myc, NF-AT, NF-KB and the like), expression of cytokine receptors, expression of chemokines, and expression of chemokine receptors. Assays suitable for detecting each of these activities, and for correlating these activities with anti-inflammatory effects, are well known in the art.

The T cells used to determine T cell regulatory activity in response to TCR peptides can be derived from any convenient T cell source, such as lymphatic tissue, spleen cells, blood, cerebrospinal fluid (CSF) or synovial fluid. The T cells can be enriched, if desired, by standard positive and negative selection methods. If enriched, the T cell population should retain a sufficient number of antigen-presenting cells to present the TCR peptide to the regulatory T cells. A convenient source of T cells to use in the assay are peripheral blood mononuclear cells (PBMC), which can be readily prepared from blood by density gradient separation, by leukapheresis or by other standard procedures known in the art.

In order to determine regulatory activity in response to a particular TCR V peptide, the T cells are stimulated with the peptide for a suitable period of time and under suitable conditions to elicit a detectable amount of the regulatory activity by the assay method used. The stimulatory peptide can contain the complete V chain, or any immunogenic portion of the V region that is characteristic of the particular TCR V gene or gene family of interest. Such a peptide can have a sequence that is identical to that of the naturally-occurring V chain, or can contain 1, 2 or several substitutions that do not alter its specificity for the TCR V gene or gene family of interest.

Useful stimulatory V peptides will generally be from about 8 to about 100 amino acids in length, such as from about 10 to about 50 amino acids, including from about 15 to about 30 amino acids. Stimulatory peptides having any amino acids sequence of interest can be prepared by methods known in the art, including chemical synthesis and recombinant methods.

The CDR2 region, which corresponds to amino acids 38-58 of AV and BV chains, is a region that is characteristic of each TCR V chain. The amino acid sequences of peptides corresponding to amino acids 38-58 of each of the 116 known AV and BV chains are shown in Tables 2 and 3. Within a given family (e.g. BV6) or subfamily (e.g. BV6S1) of V chains, amino acids 38-58 generally differ at only one or several positions. Accordingly, if desired, a consensus CDR2 peptide can be prepared, which does not necessarily have the exact sequence of any naturally occurring V chain, but which stimulates T cells that are reactive against all members of the family or subfamily.

Appropriate stimulatory peptides to use in the invention methods can be determined by those skilled in the art. The immunogenicity of a given peptide can be predicted using well-known algorithms that predict T cell epitopes (see, for example, Savoie et al., Pac. Symp. Biocomput. 1999:182-189 (1999); Cochlovius et al., J. Immunol. 165:4731-4741 (2000)). Both the immunogenicity and the specificity of a given peptide can be confirmed by standard immunological assays that measure in vivo or in vitro T cell responses (e.g. T cell proliferation assays, delayed type hypersensitivity assays, ELISA assays, ELISPOT assays and the like).

The invention methods generally involve an initial comparison between T cell regulatory activity in response to a TCR V peptide in a test individual and a normal value for the same regulatory activity. The normal value can be a value obtained from a single healthy control individual, but will preferably be an average of values obtained from a number of healthy control individuals. Suitable healthy control individuals can be determined by the skilled person, but generally will be appropriately matched for age, gender and other variables that can affect immunological activity. The normal value for regulatory activity can be determined at the same time, prior to or after assaying for regulatory activity in the test individual.

As used herein, the term “low” with respect to a particular T cell regulatory activity refers to an activity that is significantly reduced in a test individual compared to the normal value for that activity. The extent of reduction required for significance will vary depending on the sensitivity and reproducibility of the method, but will generally be at least 25% lower than a normal value obtained for the same activity or response, such as at least 40%, 50%, 70%, 80% or 90% lower than the normal value. The term “low” also includes a complete absence of detectable activity, as evidenced by a background level of activity.

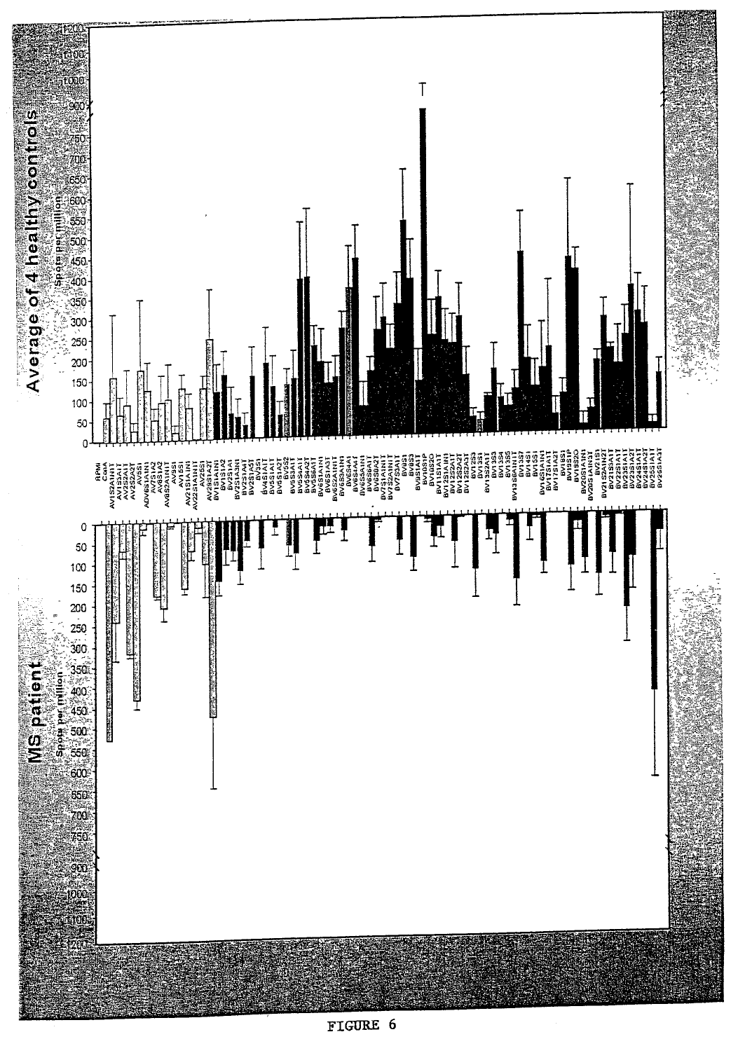

As an example, FIG. 6 shows a comparison of the number of T cells per million that secrete the regulatory cytokine IL-10 in response to a panel of AV and BV peptides in an MS patient and in healthy controls. By the above definition, the MS patient exhibits essentially normal T cell regulatory activity in response to stimulation by ConA and most AV peptides, and exhibits “low” T cell regulatory activity in response to the majority of VB peptides.

Changes in T cell regulatory activity in a single individual can be monitored over time to determine development or progression of an autoimmune disease, to monitor the efficacy of a therapy in restoring normal regulatory activity, or to determine an appropriate time to initiate, stop or readminister a therapy to boost regulatory activity. In performing such comparative assays, T cell samples obtained at various times can be frozen, and multiple assays performed simultaneously to minimize experimental variables. Assays can also be repeated several times and values averaged to increase the significance of observed differences.

As described above, another component of the mechanism underlying autoimmune disease is unregulated expansion of autoreactive T cells. These T cells have escaped normal regulation by V-specific regulatory T cells, and will preferentially express a corresponding V gene or limited set of V genes.

As used herein, the term “preferentially expressed” indicates that the particular TCR gene is expressed at a significantly higher level among activated T cells in an individual than among unselected T cells from the same individual. The term “unselected T cells” encompasses any T cell population that has not been preselected for activated T cells, or which is not expected to be enriched (in comparison with PBMCs) for activated T cells. Exemplary populations of unselected T cells include, for example, peripheral blood mononuclear cells and CD4+ enriched blood cells. The level of enhanced TCR V gene expression required for significance, and thus for “preferential expression,” will vary depending on the sensitivity and reproducibility of the method, but will generally be at least a 20% increase, such as a 30%, 40%, 50%, 75%, 100% or greater increase in expression in the activated population than in the unselected T cell population.

As used herein, the term “activated T cells” refers to CD4+ T cells that have undergone characteristic phenotypic and functional changes as a result of interacting with antigen presented in the context of class II MHC. Such phenotypic and functional changes can include, for example, expression of activation surface markers, secretion of Th1 cytokines, and proliferation.

Activation surface markers include CD25, which is the IL-2 receptor, CD134 (OX-40), which is a cell surface glycoprotein in the tumor necrosis factor receptor family, as well as CD30, CD27 and CD69. The structural and functional properties of T cell activation surface markers, as well as reagents suitable for detecting such markers, are well known in the art (see, for example, Barclay et al., “The Leucocyte Antigen FactsBook,” Academic Press, San Diego, Calif. (1993)).

Activated T cells can further express surface marker profiles characteristic of memory T cells, which include, for example, expression of CD45R0+ and lack of expression of CD45RA. Therefore, in one embodiment, the method in practiced by determining TCR V gene expression among activated, memory T cells.

Secreted cytokines that are characteristic of activated CD4+ T cells include, for example, interleukin-2 (IL2), IL4, IL5, and γ-interferon (IFNγ). The structural and functional properties of various cytokines, as well as reagents suitable for detecting cytokine expression and secretion, are well known in the art (see, for example, Thomson, ed., “The Cytokine Handbook,” 2^{nd} ed., Academic Press Ltd., San Diego, Calif. (1994)).

A population of cells that contains activated T cells can be obtained from a variety of sources, including the peripheral blood, lymph, and the site of the pathology. The peripheral blood is generally the most convenient source of cells. However, appropriate pathological sites include the CNS (and particularly the cerebrospinal fluid) for multiple sclerosis and other autoimmune neurological disorders; the synovial fluid or synovial membrane for rheumatoid arthritis and other autoimmune arthritic disorders; and skin lesions for psoriasis, pemphigus vulgaris and other autoimmune skin disorders, any of which can be readily obtained from the individual. As available, biopsy samples of other affected tissues can be used as the source of T cells, such as intestinal tissues for autoimmune gastric and bowel disorders, thyroid for autoimmune thyroid diseases, pancreatic tissue for diabetes, and the like.

The cell population need not be pure, or even highly enriched for activated T cells, so long as the method allows for a comparison of TCR gene expression by activated and unselected T cells. For example, by FACS analysis the expression of both an activation surface marker and a V chain polypeptide can be detected simultaneously, without enrichment for activated T cells, and the number of activated and non-activated (or total) T cells expressing the V chain compared.

Depending on the assay method, it may be desirable to start with a cell population that is partially enriched, or highly enriched, for activated T cells. Methods for enriching for desired T cell types are well known in the art, and include positive selection for the desired cells, negative selection to remove undesired cells, and combinations of both methods.

Enrichment methods are conveniently performed by first contacting the cell population with a binding agent specific for a particular T cell surface activation marker or combination of markers. Appropriate binding agents include polyclonal and monoclonal antibodies, which can be labeled with a detectable moiety, such as a fluorescent or magnetic moiety, or with biotin or other ligand. If desired, the T cells can be further contacted with a labeled secondary binding agent specific for the primary binding agent. The bound cells can then be detected, and either collected or discarded, using a method appropriate for the particular binding agent, such as a fluorescence activated cell sorter (FACS), an immunomagnetic cell separator, or an affinity column (e.g. an avidin column or a Protein G column). Other methods of enriching cells by positive and negative selection are well known in the art.

As described in Example I, by immunomagnetic separation to remove non-T cells, and FACS sorting to enrich for activated T cells, CD4+,CD25+,CD45RA- cells were enriched to about 83-95% purity. Similar methods can be used for enriching for T cells that express other activation surface markers.

Analogous methods have recently been developed for enriching for cells that secrete activation cytokines. In such methods a bivalent binding agent (e.g. a bivalent antibody) with specificity for both the secreted molecule and a cell surface molecule are allowed to contact the T cells. The secreted molecule, now relocated to the affinity matrix, is then contacted with a binding agent and bound cells sorted or separate by standard methods (see, for example, WO 99/58977 and Brosterhaus et al., Eur. J. Immunol., 29:4053-4059 (1999)).

TCR V gene expression by the selected or unselected T cell population can be determined by a variety of methods. For example, such methods can be based on detection and quantification of expressed TCR V polypeptide chains, TCR V gene transcripts, or rearranged V genes.

Detection and quantification of V polypeptide expression can be practiced using agents that specifically bind particular V polypeptides, such as anti-V chain antibodies. Antibodies specific for a variety of Vα, Vβ, Vγ, and Vδ chains are available in the art (see, for example, Kay et al., Leuk. Lymphoma 33:127-133 (1999); Mancia et al., Scand. J. Immunol. 48:443-449 (1998)). Alternatively, suitable polyclonal or monoclonal antibodies can be prepared by standard methods (see, for example, Harlow and Lane, Antibodies: A laboratory manual (Cold Spring Harbor Laboratory Press 1988); Ausubel et al., Current Protocols in Molecular Biology, John Wiley and Sons, Baltimore, Md. (2001)), starting from a V chain peptide.

Methods of detecting V polypeptide expression can be practiced using either whole cells or cell extracts. For example, whole cells can be contacted with appropriate detectably labeled antibodies and/or detectably labeled secondary antibodies. Cells that specifically bind the particular anti-V antibody are then detected and quantified by standard methods appropriate for the particular detectable label, such as FACS or immunofluorescence microscopy for fluorescently labeled molecules, scintillation counting for radioactively labeled molecules, and the like. Alternatively, cell extracts can be contacted with appropriate anti-V antibodies, and V polypeptide expression analyzed using standard methods, such as immunoprecipitation, immunoblotting or ELISA (see, for example, Ausubel et al., Current Protocols in Molecular Biology, John Wiley and Sons, Baltimore, Md. (2001)).

Methods for detecting and quantifying TCR V gene transcripts or rearranged V genes generally involve specific hybridization of nucleic acid probes or primers to MRNA, cDNA, or genomic DNA, as appropriate, from the T cells of interest. The nucleotide sequences of Vα, Vβ, Vγ, and Vδ genes are well known in the art (see, for example, Genevee et al., Eur J. Immunol. 22:1261-1269 (1992); Arden et al., Immunogenetics 42:455-500 (1995); Choi et al., Proc. Natl. Acad. Sci. USA 86:8941-8945 (1989); Concannon et al., Proc. Natl. Acad. Sci. USA 83:6598-6602 (1986); Kimura et al., Eur J Immunol. 17:375-383. (1987); Robinson, J. Immunol. 146: 4392-4397 (1991); and the EMBL alignment database under alignment accession number DS23485). Therefore, the skilled person can readily prepare probes and primers specific for any TCR V gene of interest, appropriate for the particular detection method.

Exemplary detection methods include, for example, reverse-transcriptase polymerase chain reaction (RT-PCR), Northern blots, RNase protection assays, in situ hybridization, and the like. Detection methods can conveniently employ radiolabeled or fluorescently labeled nucleotides, such that the amount of hybridization or amount of amplified product can be detected by a commercially available phosphorimaging apparatus. Suitable methods for detecting and quantitating mRNA expression are described, for example, in Ausubel et al., supra (2001) and other standard molecular biology manuals.

Example I shows the use of RT-PCR to quantitate expression of members of 24 Vβ families in different sorted populations of CD4+ T cells.

Automated assays for simultaneously detecting and quantitating expression of a plurality of genes are also well known in the art, and are contemplated herein for determining V gene expression. For example, nucleic acid molecules specific for all or a particular subset of V genes can be attached to a solid support, such as a plate, slide, chip or bead, which can then be contacted with the appropriate T cells, T cell extracts, or T cell nucleic acid molecules, under suitable hybridization conditions, and processed automatically by standard methods. Likewise, immunological assays for simultaneously detecting expression of a plurality of polypeptides are well known in the art and are contemplated herein. Such methods generally involve the use of a plurality of different antibodies bound to a solid support, and binding can be detected by automated detection systems.

The invention also provides kits that contain reagents for use in identifying V gene expression by a target T cell population by the methods described above. As use herein, the term “kit” refers to components that are packaged together, in a single container or separate containers, or otherwise indicated to be for use together. An indication that the components are to be used together can be, for example, written instructions to this effect, such as written instructions for determining TCR V gene expression and for determining regulatory T cell activity using the kit components.

In one embodiment, the kit contains at least one TCR V peptide suitable for eliciting a regulatory T cell response, and at least one agent suitable for detecting TCR V gene expression. Suitable TCR V peptides have been described above. Suitable agents for detecting TCR V gene expression, which include anti-V chain antibodies and V gene-specific hybridization probes and primers, have also been described above.

Optionally, the kit can further contain reagents (e.g. buffers, enzymes, antibodies, detection reagents and the like) and/or supplies (e.g. microwell plates, tubes and the like) suitable for performing some or all of the following procedures: obtaining T cell samples from the individual; sorting or enriching for activated T cells; isolating PBMCs; performing nucleic acid hybridization or amplification procedures; performing immunospot or ELISPOT procedures; and analyzing the data so obtained. For example, a kit can further contain microtiter plates, antibodies and detection reagents suitable for detecting IL-10 secretion by activated T cells in an immunspot assay.

A kit can contain at least one (such as 2, 5, 10, 15, 20, 30, 50, 75, 100, 116 or more) TCR V peptides. For example, a kit can contain any or all of the 51 AV and 65 BV CDR2 peptides shown in Tables 2 and 3, or any subset thereof. Likewise, the kit can contain at least one (such as 2, 5, 10, 15, 20, 30, 50, 75, 100, 116 or more) V-specific hybridization probes, V-specific PCR primers or anti-V antibodies. For example, a kit can contain any or all of the AV and BV PCR primers shown in Tables 4 and 5, or any subset thereof. At least one of the V peptides and one of the V-specific agents will correspond to each other.

The number and type of V peptides and agents for detecting V gene expression to be included in the kit will depend on the intended application. For example, for initially identifying a target T cell in an individual, a kit can contain a complete, or relatively complete, set of V peptides and V agents specific for known AV, BV, Vγ and/or Vδ chains. However, for determining whether an individual is likely to respond to a particular therapy for autoimmune disease, a kit can optionally contain only those reagents that correspond to the V genes targeted by the particular therapy.

As an example, to determine whether a therapeutic product that targets Vβ3, Vβ14 and/or Vβ17 T cell receptors, including immunogenic peptides, nucleic acid molecules and cytotoxic agents (as described in U.S. Pat. Nos. 6,090,387; 6,221,352; 6,159,470; and 5,837,246) is likely to be effective for treating rheumatoid arthritis in a particular individual, the kit can contain Vβ3, Vβ14 and/or Vβ17 peptides together with agents suitable for detecting TCR Vβ3, Vβ14 and/or Vβ17 gene expression, as set forth in Tables 3 and 5.

As a further example, to determine whether a therapeutic product that targets Vβ3, Vβ13.1 and/or Vβ17 T cell receptors, including immunogenic peptides, nucleic acid molecules and cytotoxic agents (as described in PCT publication WO 95/19375) is likely to be effective for treating psoriasis in a particular individual, the kit can contain Vβ3, Vβ13.1 and/or Vβ17 peptides together with agents suitable for detecting TCR Vβ3, Vβ13.1 and/or Vβ17 gene expression, as set forth in Tables 3 and 5.

As another example, to determine whether a therapeutic product that targets Vβ2, Vβ5, Vβ6, Vβ7 and/or Vβ13 T cell receptors, including immunogenic peptides, nucleic acid molecules and cytotoxic agents (as described in PCT publication WO 94/25063 and U.S. Pat. Nos. 5,776,459 and 5,614,192) is likely to be effective for treating multiple sclerosis in a particular individual, the kit can contain Vβ2, Vβ5, Vβ6, Vβ7 and/or Vβ13 peptides together with agents suitable for detecting TCR Vβ2, Vβ5, Vβ6, Vβ7 and/or Vβ13 gene expression, as set forth in Tables 3 and 5.

As yet another example, to determine whether a therapeutic product that targets Vβ6 and/or Vβ14 T cell receptors, including immunogenic peptides, nucleic acid molecules and cytotoxic agents (as described in U.S. Pat. No. 6,113,903) is likely to be effective for treating diabetes in a particular individual, the kit can contain Vβ6 and/or Vβ14 peptides together with agents suitable for detecting TCR Vβ6 and/or Vβ14 gene expression, as set forth in Tables 3 and 5.

The skilled person can apply the knowledge of the TCR V genes expressed by a target T cell populations in an individual to a variety of diagnostic, prognostic and therapeutic applications. For example, one can determine the effect of established or new therapies either on increasing T cell regulatory activity toward the target T cells, or on reducing the number of target T cells (as evidenced by a reduction in expression of the V gene by activated T cells). A therapy that restores more normal immunoregulation of the target T cell population is expected to also be effective in reducing the clinical symptoms of the disease.

Likewise, by monitoring the T cell regulatory activity toward the target T cells, or the number of target T cells during therapy, a judicious decision can be reached regarding when and whether to initiate, readminister or terminate the therapy, thereby reducing potential side effects caused by ineffective or unnecessary therapy.

A variety of therapies have been proposed or are currently in use for treating autoimmune diseases. The methods and kits of the invention can be used in conjunction with any of these therapies.

For example, therapies that selectively target populations of T cells expressing particular TCR V chains have been developed. These therapies include immunogenic TCR pepides and TCR peptide-encoding nucleic acid molecules that induce a regulatory immune response against the particular T cell, and anti-TCR specific cytotoxic agents that bind to and kill or inhibit the activity of the particular T cell (see, for example, U.S. Pat. Nos. 5,614,192 and 5,612,035).

Likewise, both immunization and immunoblocking methods that target HLA molecules associated with autoimmune diseases are being developed as therapies. For example, expression of HLA-DR1 and some subtypes of HLA-DR4 (eg. Dw4) are strongly associated with rheumatoid arthritis (RA); expression of HLA-B27 is strongly associated with ankylosing spondylitis and reactive arthritis; expression of HLA-DR15, DQ6 and Dw2 with multiple sclerosis (MS); HLA-DR3 and HLA-DR4 with diabetes; and HLA-DR2 and HLA-DR3 with lupus. The association of HLA haplotypes with immune pathologies and methods of targeting HLA molecules are described, for example, in U.S. Pat. No. 6,045,796.

Other therapies currently in use or proposed for treating autoimmune diseases include inducing tolerance towards known or presumptive autoantigens. Autoantigens and the use of autoantigens to induce tolerance are known in the art and described, for example, in U.S. Pat. Nos. 6,039,947; 6,019,971; 5,869,093; 5,858,968 and 5,856,446. Known or suspected autoantigens, with their associated diseases, include: myelin basic protein, proteolipid protein, myelin oligodendrocytic glycoprotein, myelin associated glycoprotein, and αB-crystallin (multiple sclerosis and EAE); collagen type II, heat shock proteins, aggrecans, proteoglycans, fillagrin and link (collagen-induced arthritis, adjuvant-induced arthritis, rheumatoid arthritis); desmin (psoriasis); S-antigen (uveitis); insulin, glutamic acid decarboxylase (NOD, type I diabetes); tropomyosin (inflammatory bowel disease); epidermal cadherin (pemphigus vulgaris); Sm, RNP, histones (systemic lupus erythematosus); thryoid stimulating hormone receptor (Grave's disease); thyroglobulin, peroxidase (Hashimoto's thyroiditis); collagen type IV (Goodpasture's syndrome); platelet integrin a IIb: IIIa (autoimmune thrombocytopenia purpura); Rh blood group 1 antigen (autoimmune hemolytic anemia); and acetylcholine receptor (myasthenia gravis).

Furthermore, therapies for autoimmune diseases include administering altered peptide ligands, which are analogs of an antigenic peptide (such as the autoantigenic peptides described above), in which the TCR contact residues have been altered, such that the peptide binds the HLA molecules with similar affinities as the wild-type peptide, but does not stimulate T cell proliferative responses. Methods of making and using altered peptide ligands of a variety of antigenic peptides are described, for example, in Evavold et al., Immunology Today 14:602-609 (1993), in Fairchild, Eur. J. Immunogenet. 24:155-167 (1997), and in U.S. Pat. No. 6,197,926.

A variety of other therapies that affect T cell activity are currently in use or are under development for treating autoimmune diseases. Such therapies include, for example, vaccines and blocking agents that target antigens present on all or most T cells (e.g. CD2, CD3, CD4, CD5, CD6, CD7, CD8, CD27, CD28, CD32, CD43, and T cell receptor constant regions); general immunosuppressive agents such as corticosteroids, cyclosporine and FK506; anti-inflammatory cytokines such as IL-4, IL-10, TGF-β and interferons (e.g. interferon (IFN)beta-1a (Avonex™); IFNbeta-1b (Betaseron™); Rebif™); agents that non-specifically interfere with TCR/HLA/antigen interactions (e.g. the basic four-amino acid copolymer glatiramer acetate (Copaxone™)); agents that bind to pro-inflammatory cytokines (e.g. Enbrel™; etanercept; infliximab); cytokine receptor antagonists (e.g. IL-1 receptor antagonist); antineoplastic agents (e.g. mitoxantrone (Novantrone™); purine analogs (e.g. 2-chlorodeoxyadenosine (cladribine); 2′-deoxycorfomycin (pentostatin)) as well as methotrexate, Cox-2 inhibitors (e.g. etoricoxib), phosphodiesterase inhibitors, leflunomide and the like, and various combinations of the above agents. Other established and potential therapies for autoimmune diseases are well known in the art.

The following examples are intended to illustrate but not limit the present invention.

### EXAMPLE I

This example shows that there is an inverse correlation between the number of regulatory, cytokine-secreting T cells specific for a particular T cell receptor V chain, and the number of autoreactive T cells bearing that V chain.

Materials and Methods

Patients. Study participants included 7 male and 17 female MS patients (ages 24-74) with definite relapsing-remitting or progressive MS, and 8 male and 7 female healthy controls (HC, ages 23-55). MS patients had diagnosed MS for 2-30 years, and were currently receiving Avonex™, Betaseron™, Copaxone™, or no treatment, but not corticosteroids. Blood samples were obtained from the MS clinic after obtaining informed consent. Three MS patients had been successfully vaccinated with the (Y49T)BV5S2-38-58 peptide in previous trials, and continued to receive monthly booster injections.

TCR V gene expression. Peripheral blood mononuclear cells (PBMCs) were obtained and enriched for CD4+ T cells by removal of B cells, monocytes, NK cells and CD8+ T-cells using antibody-coated magnetic beads. These cells were then stained with fluorescent mAb specific for CD4, activation (CD25), and naive (CD45RA) T cell markers. CD4+ cells were gated and sorted by FACS to obtain activated memory T-cells (CD25+, CD45RA−), as well as non-activated naive T-cells (CD25−, CD45RA+). mRNA was prepared from the CD4+ starting population, activated memory cells, and resting naive T-cells, and evaluated for V gene expression by RT-PCR, essentially as described in Chou et al., J. Immunol. 152:2520-2529 (1994), using BV gene specific primers set forth in Table 5.

Briefly, total RNA was isolated from fresh pelleted cells using the Stratagene RNA Isolation Kit (Stratagene, La Jolla, Calif.). cDNA was synthesized in a 20 μL volume using Superscript II reverse transcriptase (Life Technologies, Rockville, Md.) and an oligo(dT)12-18primer (Life Technologies, Rockville, Md.) following the manufacturer's recommendations. For amplification of TCRBV cDNA, a panel of 26 BV and a single BC primer was used. A portion of the BC primer was labeled (either 2 to 3% was radioactively labeled with ³²P-ATP, or 50% was end labeled at the 5′ end with the fluorochrome, Cy3 (Amersham Pharmacia Biotech, Piscataway, N.J.). As a positive control for the reaction, two BC primers (forward and reverse) were used, and the reverse primer was labeled as above. The CDNA from 1500 to 2000 T cells was used in each 15 μL reaction, along with 0.3 μL of each primer, 0.5U Taq DNA polymerase (Promega, Madison, Wis.), 50 mM KCl, 10 mM Tris-HCl (pH 9), 0.1% Triton X-100, 0.2 mM dNTPs, and 2 mM MgCl2. Amplification was carried out for 24-26 cycles (94.5 C×30 sec, 60 C×1 min, 72 C×1 min), followed by a final 5 min extension at 72 C. All PCR reactions were performed in a Perkin Elmer GeneAmp 9600 thermocycler (Perkin-Elmer, Norfolk, Conn.).

For the amplification of TCRAV cDNA, a panel of 30 AV primers and a AC primer were used (the AC primer was partially labeled as above). As a positive control for the reactions, two AC primers (forward and reverse) were used, one labeled as above. PCR conditions were as described above. Following amplification, 10 μL of each reaction was loaded on a 6% polyacrylamide gel and run at 250V for 22 min. If the DNA was radioactively labeled, the gel was dried for one hour, exposed to a phosphor screen for 30 min to 1 hour, and analyzed by phosphor imaging (BioRad Molecular Imager FX, BioRad, Hercules, Calif.). If the DNA was fluorescently labeled, the gel was directly imaged on a fluorescent imager (BioRad Molecular Imager FX, BioRad, Hercules, Calif.). In either case, the PCR products of the correct size were quantitated by measuring phosphor or fluorescent signal intensity, and the background subtracted using an adjacent region below the bands.

Antigens Antigens used in the ELISPOT assay included ConA (2 μg/ml) and synthetic TCR peptides (25 μg/ml), including BVSS2-38-58 (ALGQGPQFIFQYYEEEERQRG (SEQ ID NO:62), (Y49T)BV5S2-38-58 (ALGQGPQFIFQTYEEEERQRG (SEQ ID NO:183)), and BV6S1-38-58 (LGQGPEFLIYFQGTGAADDSG (SEQ ID NO:184)). Peptides were obtained from batches made for previous clinical trials, or were locally synthesized and purified by HPLC.

T cell frequency. To determine antigen-specific T cell frequency by ELISPOT, blood mononuclear cells were separated by Ficoll density gradient centrifugation, resuspended in 2% human AB serum, and aliquotted at 0.5 and 0.25 million cells in triplicate wells of nitrocellulose-coated microtiter plates (Resolution Technologies) pretreated with anti-IFN-γ ((Mabtech, Sweden)or anti-IL-10 (PharMingen, San Diego, Calif.) mAb. Peptides, ConA, and medium were added and the plates incubated at 37 C for 24 hr (IFN-γ) or 48 hr (IL-10). Biotin-labeled secondary mAb for each cytokine was added, followed by streptavidin-alkaline phosphatase (Dako Corp, Carpinteria, Calif.) and substrate (BCIP/NBT phosphatase substrate, KPL, Gaithersburg, Md.) to develop optimal blue staining. Cytokine spots were quantified using an AID Immunospot Analyzer (AID, Cleveland, Ohio) equipped with a high resolution lens camera and analytical software designed for use with the AID system. Mean spots/well were calculated for each Ag, and net counts established after subtraction of background (no Ag). The frequency of Ag-specific spot-forming cells per million PBMC was determined from the average net response observed at two different cell concentrations. The mean net frequency ±SEM was calculated for MS patients and HC, and differences compared by Student's t test for significance (p<0.05).

Results

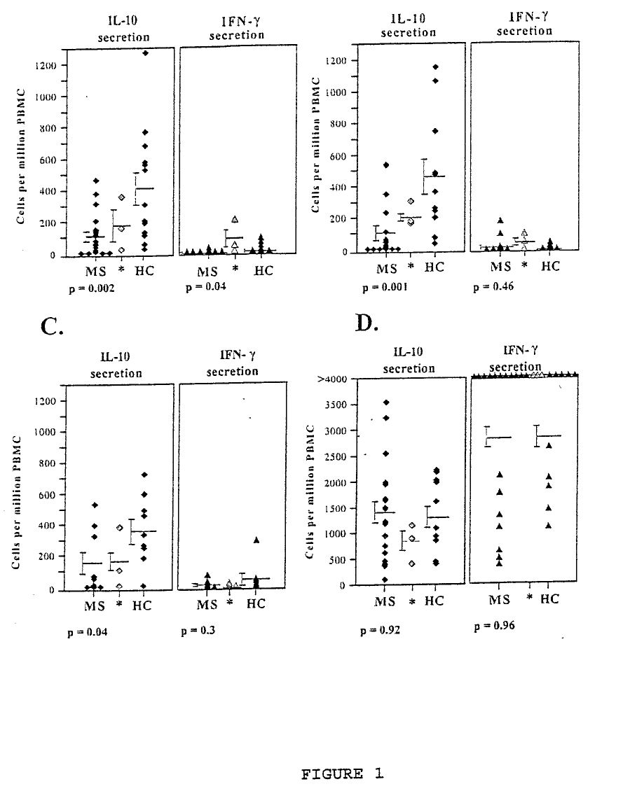

Diminished frequencies of TCR-reactive cells in MS patients versus HC: Innate frequencies of blood mononuclear cells responsive to TCR peptides, including BV5S2-38-58, (Y49T)BV5S2-38-58, and BV6S1-38-58, ConA (positive control), or no antigen (background) were quantified in MS patients and HC by ELISPOT. As is shown in Table 1 and FIG. 1, most HC had robust frequencies of IL-10 secreting cells to each of the TCR peptides tested (about 400 cells/million PBMC above background), but much lower frequencies of IFN-y secreting cells (20-50 cells/million PBMC). In contrast, MS patients had significantly lower frequencies of IL-10 secreting cells to all three TCR peptides (100-150 cells/million PBMC), with more than half of the patients having low (<50 cells/million) or absent responses. MS patients also had a lower frequency of IFN-γ secreting cells to the BV5S2-38-58 peptide. Background responses of IL-10 and to a lesser extent IFN-γ secreting cells were also decreased in MS patients compared to HC. However, frequencies of IL-10 and IFN-γ secreting cells in response to ConA were nearly identical between the two groups, indicating that MS patients were not generally immunosuppressed. No significant differences in frequencies were noted among MS patients based on age, gender, disability, or treatment status.

TABLE 1
CELLS/MILLION PBMC (background subtracted)
A. IL-b secreting cells.

| | | | (Y49T) | | |
|---|---|---|---|---|---|
| Patient | | BV5S2-38- | BV5S2-38- | BV6S1-38- | |
| Group | No Ag | 58 | 58 | 58 | Con A |
| HC Range | 17-989 | 22-1271 | 34-1152 | 0-720 | 438-2219 |
| Mean ± SEM | 396 ± 89 | 415 ± 96 | 463 ± 109 | 365 ± 70 | 1384 ± 214 |
| N | 13 | 13 | 11 | 9 | 10 |
| MS Range | 1-812 | 0-465 | 0-541 | 0-534 | 99-3535 |
| Mean ± SEM | 202 ± 48 | 118 ± 30 | 105 ± 41 | 153 ± 65 | 1416 ± 210 |
| N | 20 | 20 | 19 | 9 | 19 |
| p values | 0.05 | 0.002 | 0.001 | 0.04 | 0.92 |

B. IFN-γ secreting cells.

| Patient | | BV5S2-38- | BV5S2-38- | BV6S1-38- | |
|---|---|---|---|---|---|
| Group | No Ag | 58 | 58 | 58 | Con A |
| HC Range | 1-127 | 0-86 | 0-42 | 3-291 | 1086-4000 |
| Mean ± SEM | 42 ± 13 | 21 ± 8 | 7 ± 4 | 58 ± 39 | 2907 ± 365 |
| | 12 | 12 | 9 | 7 | 10 |
| MS Range | 2-64 | 0-30 | 0-176 | 0-72 | 378-4000 |
| Mean ± SEM | 21 ± 5 | 6 ± 2 | 18 ± 10 | 18 ± 8 | 2877 ± 343 |
| N | 18 | 18 | 18 | 8 | 18 |
| p values | 0.10 | 0.04 | 0.46 | 0.30 | 0.96 |

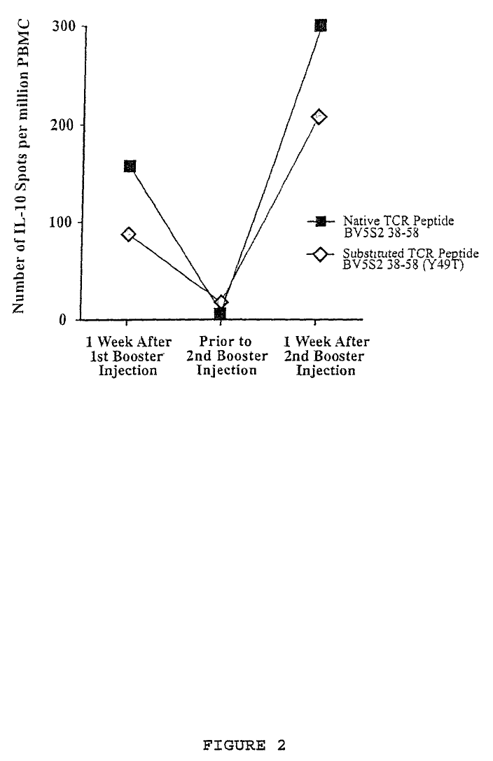

IL-10 ELISPOT responses increased in vaccinated MS patients. In previous studies, about half of the MS patients injected with (Y49T) BV5S2-38-58 peptide developed a significantly elevated frequency of proliferating peptide-responsive T-cells (Vandenbark et al., Nature Med. 2:1109-1115 (1996)). Three of these peptide-responsive patients who continued to receive monthly booster injections of (Y49T)BV5S2-38-58 peptide had moderate frequencies of IL-10-secreting cells (160-200 cells/million) as detected by ELISPOT assay, but lower frequencies of IFN-γ-secreting cells (3-85 cells/million) to the immunizing (Y49T)BV5S2-38-58 peptide, with variable responses to the other peptides and normal responses to ConA (FIG. 1, asterisks). In one patient still receiving TCR therapy, responses to Y49T-substituted or native BV5S2-38-58 peptides were boosted one week after peptide vaccination (FIG. 2). This patient had received monthly injections of (Y49T) BV5S2-38-58 peptide for about 2 years prior to the assay. Taken together, these data suggest that TCR peptide vaccination can restore TCR responses to the low normal range in responsive MS patients.

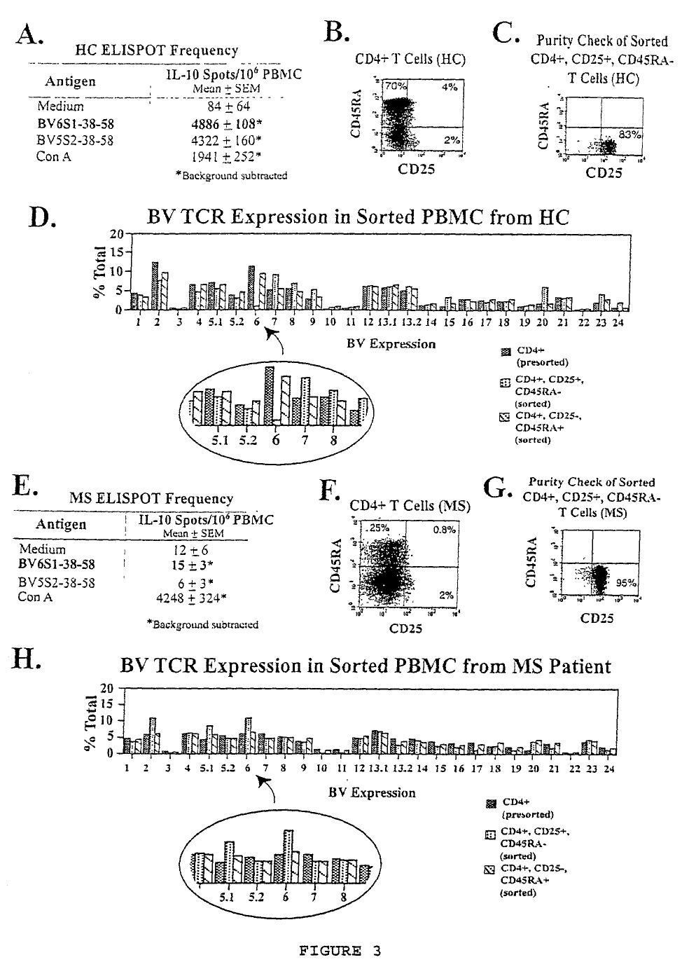

Inverse relationship between BV gene expression and TCR peptide recognition. The low innate frequencies of TCR reactive cells in MS patients suggested a regulatory deficiency that might allow expansion of potentially pathogenic memory T-cells expressing the cognate BV genes. Thus, V gene expression was evaluated in selected T cell subsets obtained by FACS sorting of blood cells. In initial experiments, ELISPOT frequencies of IL-10-secreting cells were established from a high-responder HC (FIG. 3A) versus a low-responder MS patient (FIG. 3E). Concomitantly, the BV gene repertoire was evaluated in CD4-enriched PBMC and several FACS-sorted subpopulations, including activated memory T-cells (CD4+, CD45RA−, CD25+) representing <3% of the CD4+ T-cells, as well as resting naive T-cells (CD4+, CD45RA+, CD25−)(FIGS. 3B and 3F). The sorted activated memory T-cells were 83-95% pure (FIGS. 3C and 3G). As is shown in FIGS. 3D and 3H, most of the V genes in activated memory T-cells were expressed at levels similar to the pre-sorted CD4 enriched cells and the non-activated naive T-cells. However, some BV genes in activated memory T-cells appeared to be abnormally expressed. In the high-responder HC, there was almost no expression of BV6 (see inset, FIG. 3D), whereas BV7 and BV20 appeared to be enriched. In contrast, in the low-responder MS patient, BV6, BV2 and BV5S1 were enriched (see inset, FIG. 3H). Thus, in the case of BV6, the BV gene expression was inversely related to recognition of the cognate BV gene peptide by IL-10 secreting T-cells. This same pattern of high IL-10 response in HC and absent response in MS was observed to the BV5S2 peptide, but with a less pronounced effect on BV5S2 expression by activated memory T-cells.

### EXAMPLE II

This example shows a method of determining the activity of regulatory T cells towards a panel of different TCR Vα (AV) and Vβ (BV) peptides.

Methods

Preparation of ELISPOT plates. Four flat bottom 96 well plates with nitrocellulose membranes were coated overnight with 4 μg/ml mouse anti-IL-10 monoclonal antibodies (Pharmingen), and an additional 4 plates were coated with 10 μg/ml mouse anti-human INFγ (Mabtech). Two hours before addition of peptides, plates were washed 3× with sterile PBS, pH 7.2, and blocked for one hour at room temperature with 10% FBS in sterile PBS.

Blood processing. Twelve tubes of blood (approximately 120 ml) were collected from healthy controls and MS patients. The blood was immediately separated over a Ficoll gradient by centrifugation for 25 minutes at 2100 rpm at 25 degrees. Peripheral blood mononuclear cells (PBMC) so obtained were washed 3× with cold RPMI and resuspended to 10×10⁶ cells per ml.

TCR peptide screens. Sterile stocks containing 1 mg/ml peptide were aliquoted among 4 sterile 96 well polypropylene blocks. Blocks were kept refrigerated for up to one month. Precoated and blocked ELIPSOT plates were washed with 1× with blocking solution and 100 μl of stimulation medium was added (5% fetal bovine serum/1% human AB serum/2 mM pyruvate, 2 mM glutamate, and 50 μg/ml penicillin/streptomycin). 10 μl of each peptide was added per well in triplicate wells. The sequence of each Vα (AV) peptide is shown in Table 2, and the sequence of each Vβ (BV) peptide is shown in Table 2. Negative control wells contained RPMI, positive control wells contained 18 μg/ml final concentration Con A. To each well human PBMC were added at a density of 2.5×10⁵ cells per well in a total of 8 plates (200×10⁶ cells per well). Plates were incubated for 24 hours for INFγ ELISPOTs and for 48 hours for IL-10 ELISPOTs.

TABLE 2

| Name | Amino Acid Sequence | SEQ ID NO: |
|---|---|---|
| AV1S1 | YPGQHLQLLLKYFSGDPLVKG | 1 |
| AV1S2A1N1T | YPNQGLQLLLKYTSAATLVKG | 2 |
| AV1S2A4T | YPNQGLQLLLKYTTGATLVKG | 3 |
| AV1S2A5T | YPNQGLQLLLKYTSAATLVKG | 4 |
| AV1S3A1T | YPNQGLQLLLKYLSGSTLVES | 5 |
| AV1S3A2T | YPNQGLQLLLKYLSGSTLVKG | 6 |
| AV1S4A1N1T | SPGQGLQLLLKYFSGDTLVQG | 7 |
| AV1S5 | HPNKGLQLLLKYTSAATLVKG | 8 |
| AV2S1A1 | YSGKSPELIMFIYSNGDKEDG | 9 |
| AV2S1A2 | YSGKSPELIMSIYSNGDKEDG | 10 |
| AV2S2A1T | YSRKGPELLMYTYSSGNKEDG | 11 |
| AV2S2A2T | YSRIGPELLMYTYSSGNKEDG | 12 |
| AV2S3A1T | DCRKEPKLLMSVYSSGNEDGR | 13 |
| AV3S1 | NSGRGLVHLILIRSNEREKHS | 14 |
| AV4S1 | LPSQGPEYVIHGLTSNVNNRM | 15 |
| AV4S2A1T | IHSQGPQYIIHGLKNNETNEM | 16 |
| AV4S2A3T | IHSQGPQNIIHGLKNNETNEM | 17 |
| AV5S1 | DPGRGPVFLLLIRENEKEKRK | 18 |
| ADV6SlAlN1 | SSGEMIFLIYQGSYDQQNATE | 19 |
| AV6S1A2N1 | SSGEMTFLIYQGSYDEQNATE | 20 |
| AV7S1A1 | HDGGAPTFLSYNALDGLEETG | 21 |
| AV7S1A2 | HDGGAPTFLSYNGLDGLEETG | 22 |
| AV7S2 | HAGEAPTFLSYNVLDGLEEKG | 23 |
| AV8S1A1 | ELGKRPQLIIDIRSNVGEKKD | 24 |
| AV8S1A2 | ELGKGPQLIIDIRSNVGEKKD | 25 |
| AV8S2A1N1T | ESGKGPQFIIDIRSNMDKRQG | 26 |
| AV9S1 | YSRQRLQLLLRHISRESIKGF | 27 |
| AV10S1A1 | EPGEGPVLLVTVVTGGEVKKL | 28 |
| AV11S1A1T | FPGCAPRLLVKGSKPSQQGRY | 29 |
| AV12S1 | PPSGELVFLIRRNSFDEQNEI | 30 |
| AV13S1 | NPWGQLINLFYIPSQTKQNGR | 31 |
| ADV14S1 | PPSRQMILVIRQEAYKQQNAT | 32 |
| AV15S1 | EPGAGLQLLTYIFSNMDMKQD | 33 |
| AV16SlA1T | YPNRGLQFLLKYITGDNLVKG | 34 |
| ADV17S1A1T | FPGKGPALLIAIRPDVSEKKE | 35 |
| AV18S1 | ETAKTPEALFVMTLNGDEKKK | 36 |
| AV19S1 | HPGGGIVSLFMLSSGKKKHGR | 37 |
| AV20S1 | FPSQGPRFIIQGYKTKVTNEV | 38 |
| AV21S1A1N1 | YPAEGPTFLISISSIKDKNED | 39 |
| AV22S1A1N1T | YPGEGLQLLLKATKADDKGSN | 40 |
| AV23S1 | DPGKGLTSLLLIQSSQREQTS | 41 |
| AV24S1 | DTGRGPVSLTIMTFSENTKSN | 42 |
| AV25S1 | DPGEGPVLLIALYKAGELTSN | 43 |
| AV26S1 | KYGEGLIFLMMLQKGGEEKSH | 44 |
| AV27S1 | DPGKSLESLFVLLSNGAVKQE | 45 |
| AV28S1A1T | QEKKAPTFLFMLTSSGIEKKS | 46 |
| AV29S1A1T | KHGEAPVFLMILLKGGEQMRR | 47 |
| AV29S1A2T | KHGEAPVFLMILLKGGEQKGH | 48 |
| AV30S1A1T | DPGKGPEFLFTLYSAGEEKEK | 49 |
| AV31S1 | YPSKPLQLLQRETMENSKNFG | 50 |
| AV32S1 | RPGGHPVFLIQLVKSGEVKKQ | 51 |

TABLE 3

| Name | Amino Acid Sequence | SEQ ID NO: |
|---|---|---|
| BV1S1A1N1 | SLDQGLQFLIQYYNGEERAKG | 52 |
| BV1S1A2 | SLDQGLQFLIHYYNGEERAKG | 53 |
| BV2S1A1 | FPKQSLMLMATSNEGSKATYE | 54 |
| BV2S1A3N1 | FPKKSLMLMATSNEGSKATYE | 55 |
| BV2S1A4T | FPKQSLMLMATSNEGCKATYE | 56 |
| BV2S1A5T | FPKKSLMQIATSNEGSKATYE | 57 |
| BV3S1 | DPGLGLRLIYFSYDVKMKEKG | 58 |
| BV4S1A1T | QPGQSLTLIATANQGSEATYE | 59 |
| BV5S1A1T | TPGQGLQFLFEYFSETQRNKG | 60 |
| BV5S1A2T | TLGQGLQFLFEYFSETQRNKG | 61 |
| BV5S2 | ALGQGPQFIFQYYEEEERQRG | 62 |
| BV5S3A1T | VLGQGPQFIFQYYEKEERGRG | 63 |
| BV5S4A1T | ALGLGLQLLLWYDEGEERNRG | 64 |
| BV5S4A2T | ALGLGLQFLLWYDEGEERNRG | 65 |
| BV5S6A1T | ALGQGPQFIFQYYREEENGRG | 66 |
| BV6S1A1N1 | SLGQGPEFLIYFQGTGAADDS | 67 |
| BV6S1A3T | SLGQGPELLIYFQGTGAADDS | 68 |
| BV6S2A1N1T | ALGQGPEFLTYFQNEAQLDKS | 69 |
| BV6S3A1N1 | ALGQGPEFLTYFNYEAQQDKS | 70 |
| BV6S4A1 | TLGQGPEFLTYFQNEAQLEKS | 71 |
| BV6S4A4T | NPGQGPEFLTYFQNEAQLEKS | 72 |
| BV6S5A1N1 | SLGQGLEFLIYFQGNSAPDKS | 73 |
| BV6S6A1T | ALGQGPEFLTYFNYEAQPDKS | 74 |
| BV6S8A2T | TLGQGSEVLTYSQSDAQRDKS | 75 |
| BV7S1A1N1T | KAKKPPELMFVYSYEKLSINE | 76 |
| BV7S2A1N1T | SAKKPLELMFVYSLEERVENN | 77 |
| BV7S3A1T | SAKKPLELMFVYNFKEQTENN | 78 |
| BV8S1 | TMMRGLELLIYFNNNVPIDDS | 79 |
| BV8S3 | TMMQGLELLAYFRNRAPLDDS | 80 |
| BV9S1A1T | DSKKFLKIMFSYNNKELIINE | 81 |
| BV10S1P | KLEEELKFLVYFQNEELIQKA | 82 |
| BV10S2O | TLEEELKFFIYFQNEEIIQKA | 83 |
| BV11S1A1T | DPGMELHLIHYSYGVNSTEKG | 84 |
| BV12S1A1N1 | DPGHGLRLIHYSYGVKDTDKG | 85 |
| BV12S2A1T | DLGHGLRLIHYSYGVQDTNKG | 86 |
| BV12S2A2T | DLGHGLRLIHYSYGVKDTNKG | 87 |
| BV12S2A3T | DLGHGLRLIHYSYGVHDTNKG | 88 |
| BV12S3 | DLGHGLRLIYYSAAADITDKG | 89 |
| BV13S1 | DPGMGLRLIHYSVGAGITDQG | 90 |
| BV13S2A1T | DPGMGLRLIHYSVGEGTTAKG | 91 |
| BV13S3 | DPGMGLRLIYYSASEGTTDKG | 92 |
| BV13S4 | DPGMGLRRIHYSVAAGITDKG | 93 |
| BV13S5 | DLGLGLRLIHYSNTAGTTGKG | 94 |
| BV13S6A1N1T | DPGMGLKLIYYSVGAGITDKG | 95 |
| BV13S7 | DPGMGLRLIYYSAAAGTTDKE | 96 |
| BV14S1 | DPGLGLRQIYYSMNVEVTDKG | 97 |
| BV15S1 | DPGLGLRLIYYSFDVKDINKG | 98 |
| BV16S1A1N1 | VMGKEIKFLLHFVKESKQDES | 99 |
| BV17S1A1T | DPGQGLRLIYYSQIVNDFQKG | 100 |
| BV17S1A2T | DPGQGLRLIYYSHIVNDFQKG | 101 |
| BV18S1 | LPEEGLKFMVYLQKENIIDES | 102 |
| BV19S1P | NQNKEFMLLISFQNEQVLQET | 103 |
| BV19S2O | NQNKEFMFLISFQNEQVLQEM | 104 |
| BV20S1A1N1 | AAGRGLQLLFYSVGIGQISSE | 105 |
| BV20S1A1N3T | AAGRGLQLLFYSIGIDQISSE | 106 |
| BV21S1 | ILGQGPELLVQFQDESVVDDS | 107 |
| BV21S2A1N2T | NLGQGPELLIRYENEEAVDDS | 108 |
| BV21S3A1T | ILGQGPKLLIQFQNNGVVDDS | 109 |
| BV22S1A1T | ILGQKVEFLVSFYNNEISEKS | 110 |
| BV23S1A1T | GPGQDPQFFISFYEKMQSDKG | 112 |
| BV23S1A2T | GPGQDPQFLISFYEKMQSDKG | 113 |
| BV24S1A1T | KSSQAPKLLFHYYNKDFNNEA | 114 |
| BV24S1A2T | KSSQAPKLLFHYYDKDFNNEA | 115 |
| BV25S1A1T | VLKNEFKFLISFQNENVFDET | 116 |
| BV25S1A3T | VLKNEFKFLVSFQNENVFDET | 117 |

Detection of cytokine producing cells. PBMC were removed from plates by washing with 3× with PBS and 3× with PBS/0.05% Tween, pH 7.6. To each well was added 100 μl of either anti-IFNγ (1 μg/ml, Mabtech) or anti-IL-10 (2 μg/ml, Pharmingen) and incubated for 4 hours at room temperature in the dark. Plates were washed 4× with PBS/Tween, then 100 μl per well of alkaline-phosphatase-conjugated streptaviden (DAKO) (1:1000 of stock) was added and plates were incubated for 45 minutes at room temperature. Plates were washed 4× with PBS/Tween and 6× with PBS, 1 minute each. 100 μl of BCIP/NBT substrate (KPL laboratories) was added and the color reaction was allowed to develop for 3-7 minutes. Plates were rinsed 3× with distilled water and dried overnight at room temperature.

Analysis of ELISPOTS. Plates were scanned with an Immunospot Reader (Cellular Technology Limited) with optimized lighting conditions and analyzed according to the predetermined parameters of sensitivity, spot size, and background. The background counts were subtracted, and data was then normalized to cytokine secreting cells per million PBMC plated.

Analysis of TCR gene expression. mRNA is obtained from T cells as described in Example I, and TCR gene expression is determined by RT PCR using the VA primers set forth in Table 4 and the VB primers set forth in Table 5.

TABLE 4

| Name | Nucleotide Sequence (5′ to 3′) | SEQ ID NO: |
|---|---|---|
| AV1 | GGCATTAACGGTTTTGAGGCTGGA | 118 |
| AV2 | 120CAGTGTTCCAGAGGGAGCCATTGT | 119 |
| AV3 | 123CCGGGCAGCAGACACTGCTTCTTA | 120 |
| AV4 | TTGGTATCGACAGCTTCACTCCCA | 121 |
| AV5 | CGGCCACCCTGACCTGCAACTATA | 122 |
| AV6 | TCCGCCAACCTTGTCATCTCCGCT | 123 |
| AV7 | GCAACATGCTGGCGGAGCACCCAC | 124 |
| AV8 | CATTCGTTCAAATGTGGGCAAAAG | 125 |
| AV8.1 | GTGAATGGAGAGAATGTGGAGC | 126 |
| AV8.2 | TGAGCAGAGGAGAGAGTGTGG | 127 |
| AV9 | CCAGTACTCCAGACAACGCCTGCA | 128 |
| AV10 | CACTGCGGCCCAGCCTGGTGATAC | 129 |
| AV11 | CGCTGCTCATCCTCCAGGTGCGGG | 130 |
| AV12 | TCGTCGGAACTCTTTTGATGAGCA | 131 |
| AV13 | TTCATCAAAACCCTTGGGGACAGC | 132 |
| AV14 | CCCAGCAGGCAGATGATTCTCGTT | 133 |
| AV15 | TTGCAGACACCGAGACTGGGGACT | 134 |
| AV16 | TCAACGTTGCTGAAGGGAATCCTC | 135 |
| AV17 | TGGGAAAGGCCGTGCATTATTGAT | 136 |
| AV18 | CAGCACCAATTTCACCTGCAGCTT | 137 |
| AV19 | ACACTGGCTGCAACAGCATCCAGG | 138 |
| AV20 | TCCCTGTTTATCCCTGCCGACAGA | 139 |
| AV21 | AGCAAAATTCACCATCCCTGAGCG | 140 |
| AV22 | CCTGAAAGCCACGAAGGCTGATGA | 141 |
| AV23 | TGCCTCGCTGGATAAATCATCAGG | 142 |
| AV24 | CTGGATGCAGACACAAAGCAGAGC | 143 |
| AV25 | TGGCTACGGTACAAGCCGGACCCT | 144 |
| AV26 | AGCGCAGCCATGCAGGCATGTACC | 145 |
| AV27 | AAGCCCGTCTCAGCACCCTCCACA | 146 |
| AV28 | TGGTTGTGCACGAGCGAGACACTG | 147 |
| AV29 | GAAGGGTGGAGAACAGATGCGTCG | 148 |
| AC | AGAGTCTCTCAGCTGGTACA | 149 |
| (Sol′n.151) | | |
| AC (HCA23) | GTC TCT CAG CTG GTA CAC GG | 150 |
| AC (5′) | GAACCCTGACCCTGCCGTGTACC | 151 |
| AC (3′) | ATCATAAATTCGGGTAGGATCC | 152 |

TABLE 5

| Name | Nucleotide Sequence (5′-3′) | SEQ ID NO: |
|---|---|---|
| BV1 | GCA CAA CAG TTC CCT GAC TTG CAC | 153 |
| BV2 | TCA TCA ACC ATG CAA GCC TGA CCT | 154 |
| BV3 | GTC TCT AGA GAG AAG AAG GAG CGC | 155 |
| BV4 | ACA TAT GAG AGT GGA TTT GTC ATT | 156 |
| BV5.1 | ATA CTT CAG TGA GAC ACA GAG AAA C | 157 |
| BV5.2.3 | TTC CCT AAC TAT AGC TCT GAG CTG | 158 |
| BV6.1.3 | AGG CCT GAG GGA TCC GTC TC | 159 |
| BV7 | CCT GAA TGC CCC AAC AGC TCT C | 160 |
| BV8 | ATT TAC TTT AAC AAC AAC GTT CCG | 161 |
| BV9 | CCT AAA TCT CCA GAC AAA GCT CAC | 162 |
| BV10 | CTC CAA AAA CTC ATC CTG TAC CTT | 163 |
| BV11 | TCA ACA GTC TCC AGA ATA AGG ACG | 164 |
| BV12 (B) | ACT GAC AAA GGA GAA GTC TCA GAT | 165 |
| BV13.1 (B) | CAC TGA CCA AGG AGA AGT CCC CAA T | 166 |
| BV13.2 (B) | CTC AGT TGG TGA GGG TAC AAC TGC C | 167 |
| BV14 | GTC TCT CGA AAA GAG AAG AGG AAT | 168 |
| BV15 | AGT GTC TCT CGA CAG GCA CAG GCT | 169 |
| BV16 | AAA GAG TCT AAA CAG GAT GAG TCC | 170 |
| BV17 (B) | CTA CTC ACA GAT AGT AAA TGA CTT | 171 |
| | TCA G | |
| BV18 | GAT GAG TCA GGA ATG CCA AAG GAA | 172 |
| BV19 | CAA TGC CCC AAG AAC GCA CCC TGC | 173 |
| BV20 | AGC TCT GAG GTG CCC CAG AAT CTC | 174 |
| BV21 (C) | TGT GGC TTT TTG GTG CAA TCC TAT | 175 |
| BV22 | GTT TTA TGA AAA GAT GCA GAG CGA | 176 |
| BV23 | ATA ATG AAA TCT CAG AGA AGT CTG | 177 |
| BV24 | GCA GAC ACC CCT GAT AAC TTC | 178 |
| BC (HCB-E) | CGT AGA ATT CGA CTT GAC AGC GGA | 179 |
| | AGT GGT | |
| BC (H3CB5) | CTG CTT CTG ATG GCT CAA ACA C | 180 |
| BC (5′) | CGCTGTCAAGTCCAGTTCTA | 181 |
| BC (3′) | TCTCTTGACCATGGCCATCA | 182 |

Results

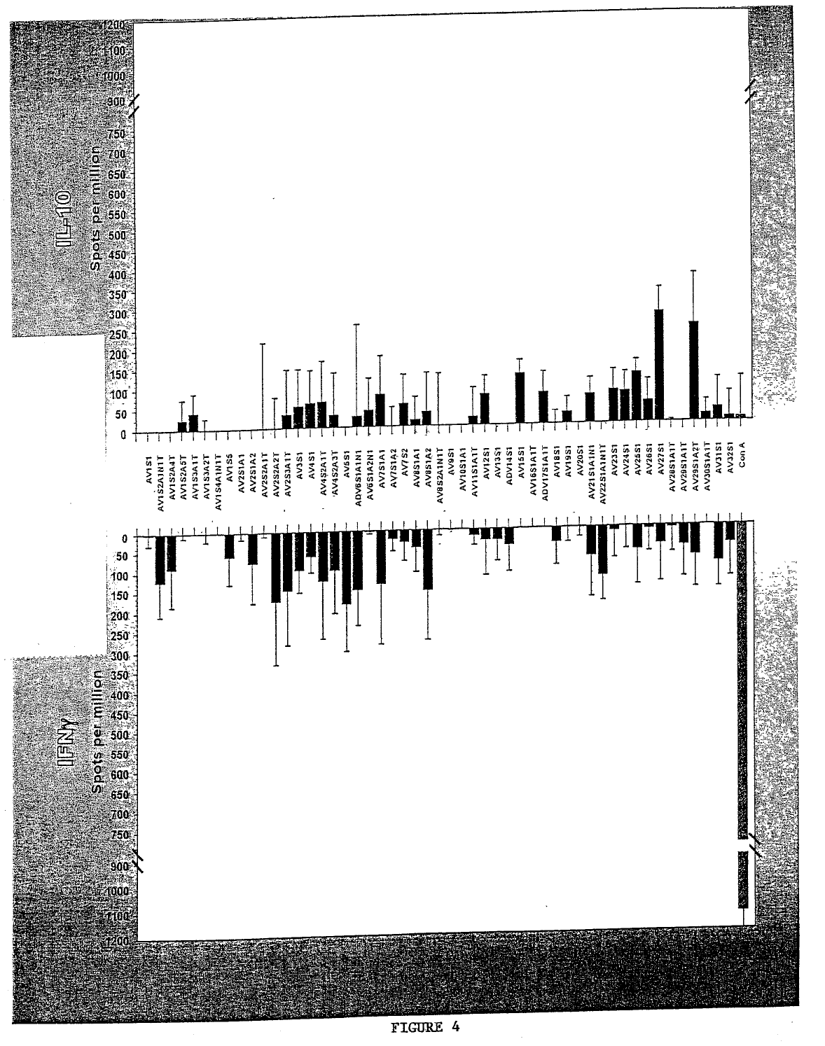

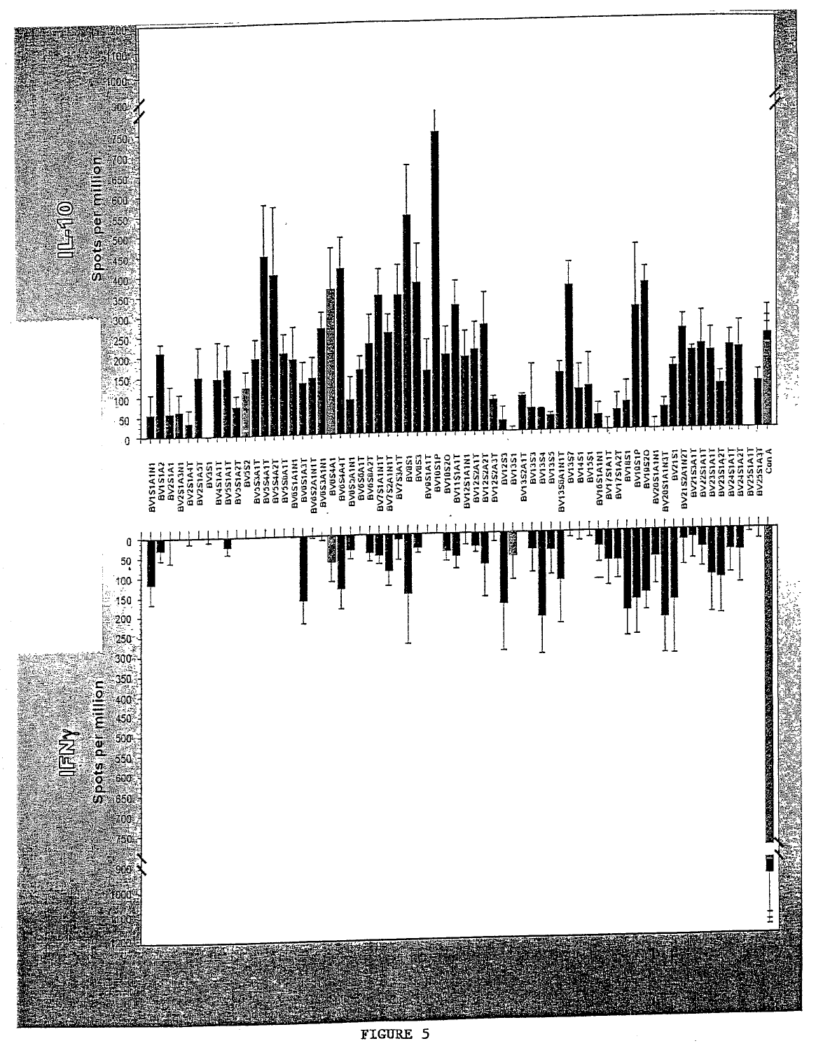

Cytokine responses to a panel of 116 different AV and BV CDR2 region peptides (amino acids 38-58) were assayed by ELISPOT in four unvaccinated, healthy individuals. FIGS. 4 and 5 show average responses to TCR AV and BV peptides, respectively, in the healthy individuals. A large number of the 116 non-redundant CDR2 seqeunces were found to be immunogenic, although generally, TCR BV peptides were more immunogenic than TCR AV peptides. Many of the immunogenic V peptides, and especially the immunogenic BV peptides, elicited an IL-10 response and little IFNγ.

FIG. 6 shows IL-10 responses to a panel of V peptides in an individual with MS (left panel), and average responses in four normal individuals (right panel). As shown in FIG. 6, in the MS patient, IL-10 responses to most Vα peptides tested were the same or higher than responses to these peptides in healthy individuals. However, responses to many Vβ peptides were significantly lower in the MS patient than in healthy individuals, and responses to certain Vβ peptides were essentially completely absent in the MS patient.

These data are consistent with there being a defect in MS patients in the immunoregulation of T cells expressing certain V chains, and particularly certain Vβ (BV) chains. It is expected that activated T cells from this individual will preferentially express a subset of these V chains, due to oligoclonal expansion of autoreactive T cells that have escaped regulation. Such cells are appropriate targets for therapeutic intervention.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention.

What is claimed is:

1. A method of identifying a T cell receptor (TCR) variable (V) gene expressed by target T cells in an individual, comprising:
a) determining expression of one or more TCR V genes by activated T cells from said individual; and
b) determining regulatory activity elicited in response to one or more TCR V peptides by T cells from said individual;
wherein a TCR V gene that is preferentially expressed in step a), whose corresponding TCR V peptide elicits low T cell regulatory activity in step b), is identified as a V gene expressed by target T cells.

2. The method of claim 1, wherein said individual has an autoimmune disease.

3. The method of claim 2, wherein said autoimmune disease is multiple sclerosis.

4. The method of claim 1, wherein expression of said one or more TCR V genes is determined by the polymerase chain reaction (PCR).

5. The method of claim 1, wherein said one or more TCR V genes are V beta genes.

6. The method of claim 1, wherein said activated T cells are characterized as CD25+CD4+ T cells.

7. The method of claim 6, wherein said activated T cells are further characterized as CD45RO+ or CD45RA− T cells.

8. The method of claim 1, wherein preferential expression of a TCR V gene is indicated by at least a 50% higher expression of said V gene in activated T cells than in unselected T cells.

9. The method of claim 1, wherein said regulatory activity is secretion of an anti-inflammatory cytokine.

10. The method of claim 9, wherein said anti-inflammatory cytokine is IL-10.

11. The method of claim 9, wherein secretion of said cytokine is determined by an immunospot assay.

12. The method of claim 1, wherein said one or more TCR V peptides are V beta peptides.

13. The method of claim 1, wherein said one or more TCR V peptides are CDR2 peptides.

14. The method of claim 1, wherein said low regulatory T cell activity in step b) is indicated by at least a 50% reduction in regulatory activity compared to a normal value.

15. A method of monitoring the efficacy of a therapy for an autoimmune disease, comprising:
a) identifying a TCR V gene expressed by target T cells in an individual with an autoimmune disease by the method of claim 1; and
b) determining T cell regulatory activity elicited in response to the corresponding TCR V peptide after initiation of therapy.

16. The method of claim 15, wherein said autoimmune disease is multiple sclerosis.

17. The method of claim 15, wherein said therapy selectively targets said T cells that express said TCR V gene.

18. The method of claim 17, wherein said therapy is immunization with a peptide corresponding to said TCR V gene.

19. A method of monitoring the efficacy of a therapy for an autoimmune disease, comprising:
a) identifying a TCR V gene expressed by target T cells in an individual with an autoimmune disease by the method of claim 1; and
b) determining expression of said V gene by activated T cells from said individual after initiation of therapy.

20. The method of claim 19, wherein said autoimmune disease is multiple sclerosis.

21. The method of claim 19, wherein said therapy selectively targets said T cells that express said TCR V gene.

22. The method of claim 21, wherein said therapy is immunization with a peptide corresponding to said TCR V gene.

23. A method of selecting a therapy for an autoimmune disease, comprising:
a) identifying a TCR V gene expressed by target T cells in an individual with an autoimmune disease by the method of claim 1; and
b) selecting a therapy that targets T cells expressing said TCR V gene.

24. The method of claim 23, wherein said autoimmune disease is multiple sclerosis.

25. The method of claim 21, wherein said therapy is immunization with a peptide corresponding to said TCR V gene.

26. A kit, comprising:
a) one or more TCR V peptides; and
b) one or more agents for detecting TCR V gene expression,
wherein said kit components are suitable for use in the method of claim 1.

27. The kit of claim 26, wherein said one or more TCR V peptides are V beta peptides.

28. The kit of claim 26, wherein said one or more TCR V peptides are CDR2 peptides.

29. The kit of claim 26, wherein said one or more agents for detecting TCR V gene expression are PCR primers.

30. The kit of claim 26, wherein said one or more agents for detecting TCR V gene expression are V beta PCR primers.

31. The kit of claim 26, comprising at least 3 TCR V peptides and at least 3 agents for detecting TCR V gene expression.

32. The kit of claim 31, comprising at least 20 TCR V peptides and at least 20 agents for detecting TCR V gene expression.
